(19) United States
(12) Patent Application Publication
(10) Pub. No.: US 2022/0383659 A1
(43) Pub. Date: Dec. 1, 2022

(54) SYSTEM AND METHOD FOR DETERMINING EMOTIONAL STATES WHEN INTERACTING WITH MEDIA

(71) Applicant: Britain Taylor, Bloomington, IN (US)

(72) Inventor: Britain Taylor, Bloomington, IN (US)

(21) Appl. No.: 17/878,498

(22) Filed: Aug. 1, 2022

Related U.S. Application Data
(60) Provisional application No. 63/195,246, filed on Jun. 1, 2021.

(51) Int. Cl. G06V 40/16; A61B 5/16

(52) U.S. Cl. CPC G06V 40/176; A61B 5/165

(57) ABSTRACT

A method for determining emotional states when interacting with media includes the steps of tracking facial expressions of a user of a media, assigning an emotional score to the tracked tracked facial expressions, and indicating negative reactions with the media based on the emotional score.

The present application claims the benefit of U.S. provisional patent application Ser. No. 63/195,246, filed Jun. 1, 2021, to Britain Taylor, titled System and Method for Determining Emotional States when Interacting with Media, the entire disclosure of which is expressly incorporated by reference herein.

### TECHNICAL FIELD

The present disclosure generally relates to a behavior regulations tracker. More particularly, the present disclosure relates to a behavior regulations tracker while a user engages on social media using computer vision and predictive analytics.

### BACKGROUND AND SUMMARY OF THE DISCLOSURE

This section introduces aspects that may help facilitate a better understanding of the disclosure. Accordingly, these statements are to be read in this light and are not to be understood as admissions about what is or is not prior art.

Almost four billion people, about half of the world's population, are active users of social media. Research has shown that young adults who use social media more than three hours per day may be at an increased risk of mental health problems. Specific content and overall consumption can have significant impacts on a user's mood and mental health—after only 30 minutes of social media consumption a user may experience low self-worth. To address these risks to a user's mood and mental health, a user could benefit from high-usage warnings, behavioral trend monitoring, and trigger identification.

According to the present disclosure, a behavior regulations tracker is provided that includes computer vision to monitor a user's facial micro-expressions and artificial intelligence to associate social media activity with the micro-expressions.

According to the present disclosure, a behavior regulations tracker is provided that monitors a user's facial micro-expressions and matches the micro-expressions with social media activity.

According to one aspect of the present disclosure, a behavior regulations tracker is provided that monitors a user's facial micro-expressions to determine behavioral trends and uses artificial intelligence to match the facial micro-expressions against the user's passive and active social media activity.

According to another aspect of the present disclosure, a behavior regulations tracker is provided that tracks internet traffic and the graphical user interface of a user's computer, tracks the facial micro-expressions of the user using the user's webcam during a social media session, the user beginning a session, tracks the content of the social media the user is engaging with and the user's facial expressions after engaging with specific content, and assigns a feedback score to specific content based on the user's facial expressions.

According to another aspect of the present disclosure, a behavioral regulations tracker is provided that tracks internet traffic and the graphical user interface of a user's computer and tracking the facial micro-expressions of a user using the user's webcam during a social media session, tracks the content of the social media the user is engaging with and the user's facial expressions after engaging with specific content, assigns a feedback score to specific content based on the user's facial expressions, stores the feedback scores, and notifies the user of the content associated with the most negative feedback scores.

### BRIEF DESCRIPTION OF THE DRAWINGS

The previously described aspects of this disclosure will grow to be appreciated at a greater level once references to the following accompanying illustrations are expounded upon.

FIG. 1 is an image of one smartphone user having a happy interaction with social media and another user having an unhappy interaction with social media;

FIG. 2 is series of images of a person showing the person expressing several different emotions including happy, neutral, angry, sad, and surprised;

FIG. 3 is a screenshot from a behavior tracker app showing real time emotion tracking based on facial recognition, where tracked emotions are assigned percentage values based on percentages of time a social media user's experiences an emotion while using social media;

FIG. 4 is a screenshot from the behavior tracker app of a graphic user interface (GUI) showing the social media user's daily happiness score while using social media with an accompanying colored circle, where the value can be compared to suggested happiness scores including below, baseline, good, or great;

FIG. 5 is a screenshot of a GUI from the behavior tracker app showing the social media user's weekly happiness score, average happiness scores for different social media platforms against a baseline happiness value of seventy, and weekly usage time on each social media platform;

FIG. 6a is a screenshot of a GUI from the behavior tracker app showing the social media user's daily emotional baseline with the emotions grouped by positive, negative, and neutral influence, with each emotion assigned a percentage value;

FIG. 6b is a screenshot of a GUI from the behavior tracker app showing media profiles and content that caused mood declines;

FIG. 7 is a screenshot of a GUI from the behavior tracker app showing media profiles that influenced a mood decline;

FIG. 8 is a screenshot of a GUI from the behavior tracker app showing an interactive dashboard which displays a monthly calendar and allows the social media user to view, on a given date, their emotional baseline, time spent within a given social media platform, and social media content that caused the user's mood to decline;

FIG. 9 is a screenshot of a warning notification from the behavior tracker app that is sent to the social media user by the behavior tracker, alerting the social media user to avoid content that produced negative emotional feedback; and

FIG. 10 is a process that the social media user and behavior tracker app follow to detect and compile data on the user's emotional behavior while engaging with social media.

The embodiments disclosed below are not intended to be exhaustive or limit the disclosure to the precise form disclosed in the following detailed description. Rather, the embodiments are chosen and described so that others skilled in the art may utilize their teachings. Unless otherwise indicated, the components shown in the figures are shown proportional to each other. It will be understood that no limitation of the scope of the disclosure is thereby intended. The disclosure includes any alterations and further modifications in the illustrative devices and described methods and further applications of the principles of the disclosure which would normally occur to one skilled in the art to which the disclosure relates.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

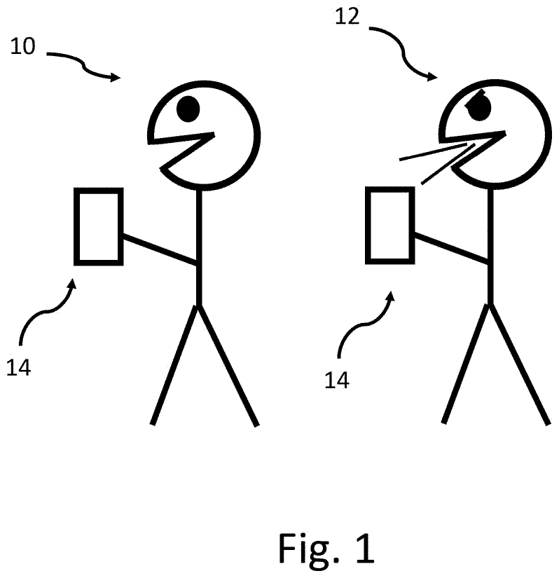

As shown in FIG. 1, two smartphone users 10, 12 are interacting with social or other media using smartphones 14. First user 10 is having a positive interaction with social media causing them to express a happy appearance. Second user 12 is having a negative interaction with social media causing them to express an unhappy appearance. Each smartphone 14 includes a behavior tracker app or other software and hardware that is configured to detect users 10, 12 emotional state and associating the emotional state with the media with which the user is interacting. The behavior tracker app also provide an emotional or happiness score to the media based on the detected emotional state. In addition to smartphones, the behavior tracker app/software may be used on other computer device, such as laptop, towers, etc. In addition to social media, the behavior tracker app/software may associate emotional states with media other than social media, such as online newspapers, chatrooms, etc.

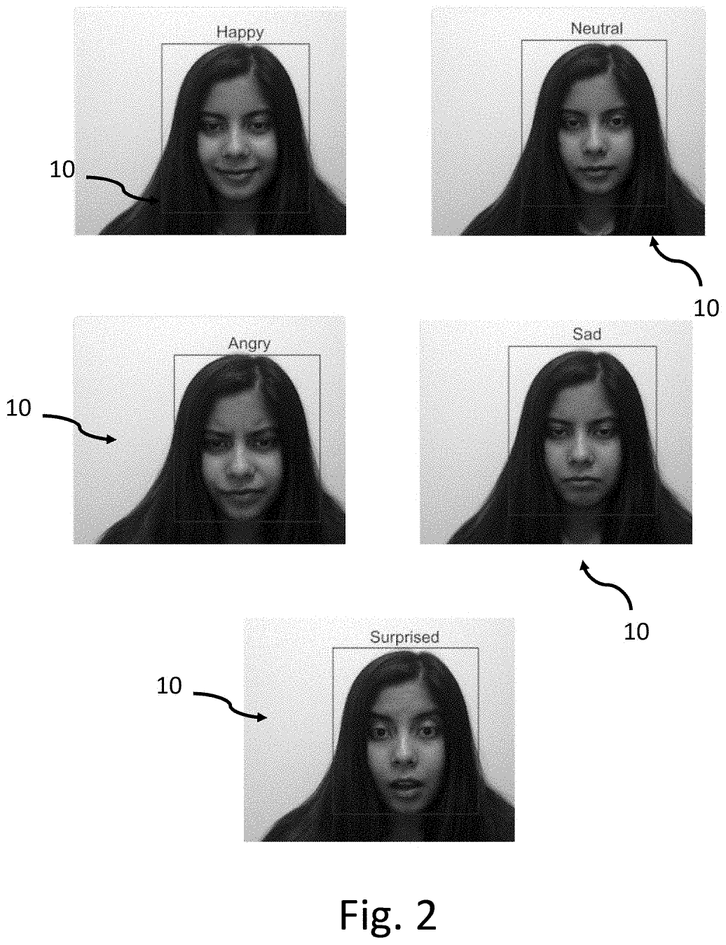

As shown in FIG. 2, people 10, 12 express their emotional state by different facial expressions. By observing the state of certain facial features, a facial expression can be identified. For example, the state of a person's mouth, eyes, and eyebrows can indicate the emotional state of persons 10, 12. As shown in FIG. 2, the emotional state of person 10 labeled “happy” is detectable by the two raised corners of their mouth. The emotional state of person 10 labeled “neutral” is detectable by the corners of the mouth being relatively flat (i.e. neither raised nor lowered). The emotional state of person 10 labeled “angry” is detectable by the lowered and drawn together eyebrows, intense staring, and/or one corner of the mouth being raised higher than the other corner. The emotional state of person 10 labeled “sad” is detectable by two lowered corners of their mouth. Finally, the emotional state of person 10 labeled “surprised” is detectable by the raised eyebrows and/or open mouth. The behavior tracker app on smartphone 14 detects these state and/or changes in these facial features to detect the user's emotional state and knows which social media platform and content of the social media platform the user in interacting with while making the facial expression. Based on the detected emotional state, social media, and content, the behavior tracker app can assess, grade/score, and track users' 10, 12 emotional reaction to various forms of social media, individual social media content providers, and specific social media content. A suitable device and method for detecting emotion is discussed in U.S. Patent No. 2015/0242679, published Jun. 13, 2017, the disclosure of which is incorporated by reference herein.

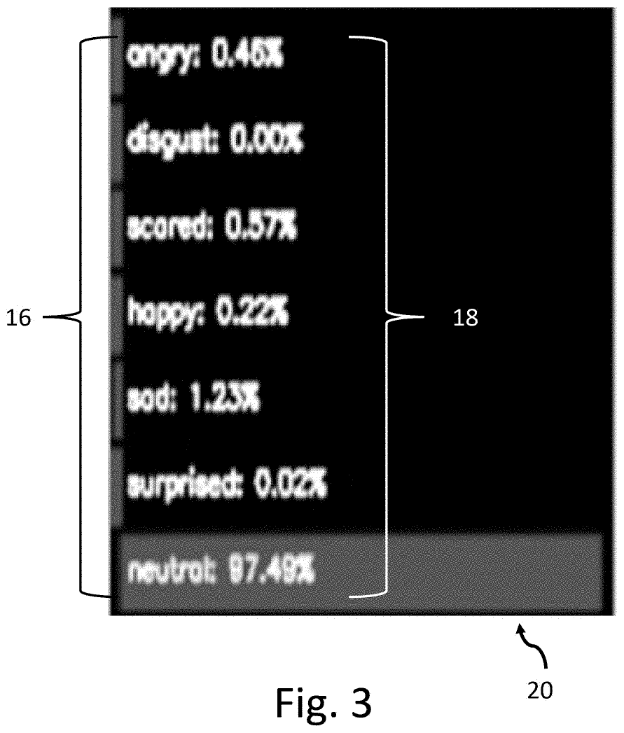

As shown in FIG. 3, the behavior tracking app shows user's 10 detected emotional responses 16 in real time. The tracker assigns percentages 18 for each emotional response 16. For example, in FIG. 3, user 10 showed angry 0.46%, disgust 0.00%, scared 0.57%, happy 0.22%, sad 1.23%, surprised 0.02%, and neutral 97.49%. FIG. 3 is a screenshot time taken during the tracker's usage. Percentages 18 allow the tracker to determine user's 10 emotional response 16 to viewed media. Percentages 18 may also be shown in bar graph form with bars 20 as shown in FIG. 3 with the neutral mode having largest bar 20.

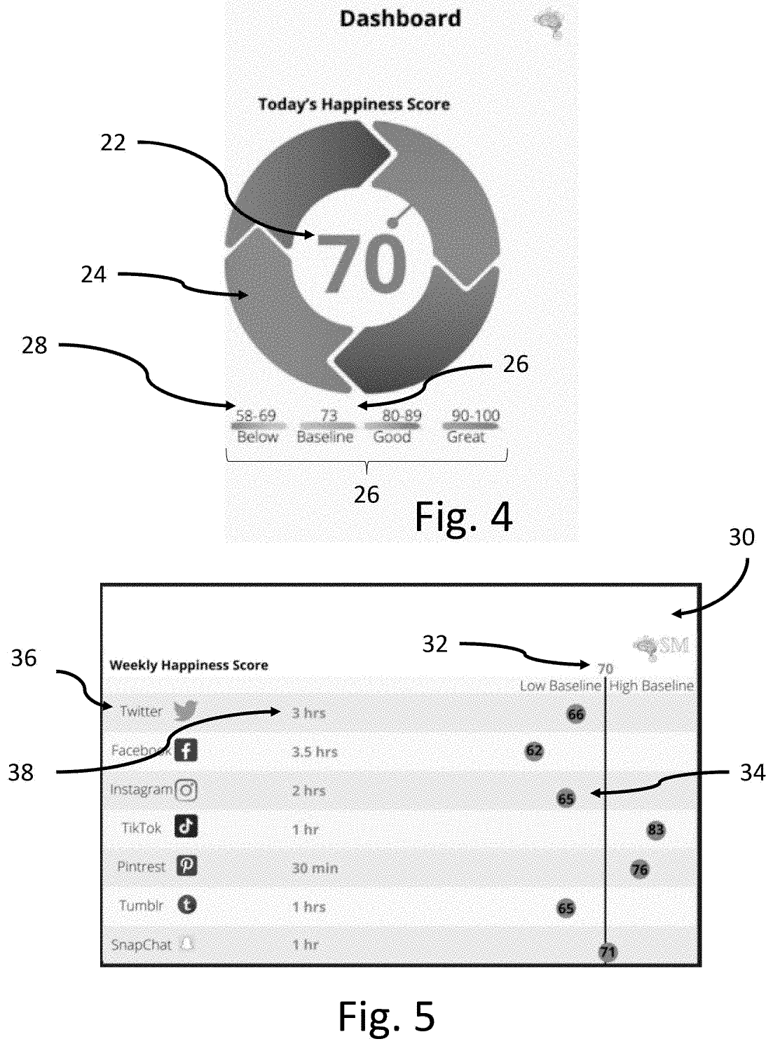

As shown in FIG. 4, a happiness score 22 is displayed by the behavior tracker app. In this example, the initial happiness score 22 is seventy (70). This initial happiness score 22 is a baseline 26 of 70. Each user's baseline 26 may be different based on each user's initial observed interactions with social media 36.

Happiness score 22 changes based on facial expressions and scores from a sentiment analysis. If user 10 expresses happy or positive expressions, happiness score 22 will increase above baseline 26 with 80 being good, 90 and above being great and an indication of overall happiness. If user 10 expresses sad, angry, or other negative emotions, happiness score 22 will decrease below baseline 26. Happiness scores 22 in the range of 50 are bad. To calculate happiness score 22, each happiness score for each type of media 36 are. The individual media scores are reflective of percentages 18 shown in FIG. 3 and FIG. 5 reflects the overall happiness score for each platform 36.

Happiness score 22 indicates to user 10 the amount of positive feedback the behavioral tracker app recorded that day. A color-coded happiness score ring 24 surrounds happiness score 22. Ring 24 graphically depicts the emotions the behavioral regulations tracker recorded that day. By reviewing ring 24, user 10 can determine what emotions were tracked and any potential patterns in the change to colors in ring 24. Within ring 24, a spectrum of color represents emotions detected above and below a baseline 26. In this example, baseline 26 is seventy-three (73), indicating that the user social media experience for the day is below baseline 26 because happiness score 22 at seventy is less than seventy-three.

In the example shown, red colors indicate score 22 is below baseline 26, blue colors indicate score 22 is above baseline 26, and a color between red and blue on the spectrum indicates score 22 is near baseline 26. Beneath happiness score ring 24, the program also displays a key 28 to correlate these colors with specific types of emotional feedback. Key 28 shows happiness score 22 and shows user 10 what colors are associated with positive, neutral, and negative emotions. Key 28 also compares these colors with baseline 26.

As shown in FIG. 5, the behavior tracking app shows user 10 a weekly happiness report 30 with a weekly happiness score 32. Report 30 breaks down happiness score 32 into different scores 34 for each social media platform 36. The behavior tracking app shows how much time 38 user 10 spent on each social media platform 36. This allows user 10 to review happiness score report 30 and determine their emotional reaction for each platform 36, along with weekly usage of each social media platform 36.

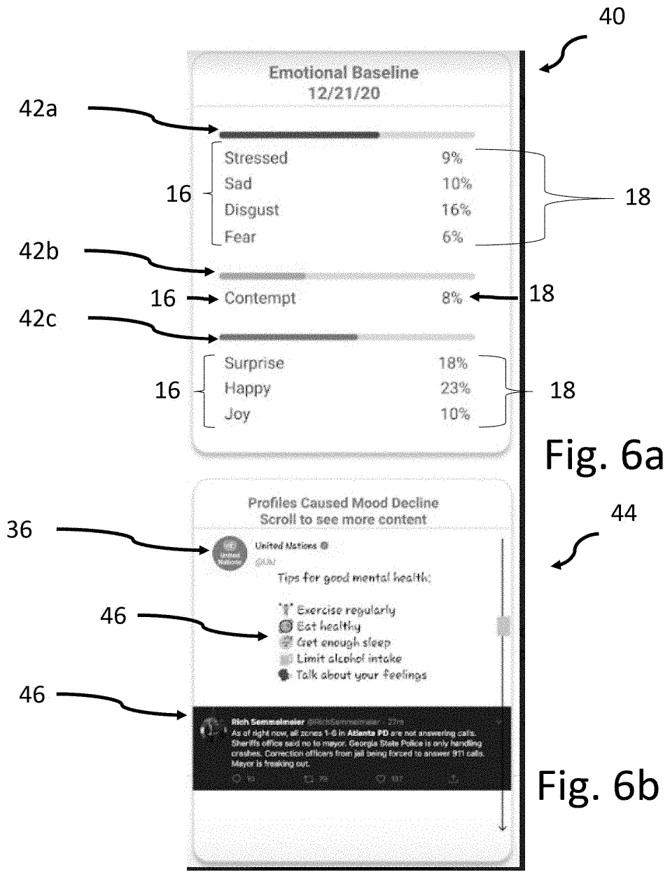

As shown in FIG. 6a, user 10 engaging with the behavior regulation tracking program on phone 14, computer, or other electronic device can access and review date specific emotional baseline reports 40 to gather information about their responses to any form of viewed media. In emotional baseline report 40, the user's emotional responses 16 that are tracked during behavioral program use are quantified as emotional percentages 18 and compiled, allowing user 10 to evaluate a comprehensive breakdown of their emotional interaction with media on a daily basis. When report 40 is viewed, similar types of emotional responses 16 are grouped together under colored bars 42 to aid user friendliness and comprehension. Negative responses 16 such as stress, sadness, disgust, and fear are organized under a red colored bar 42a. Neutral responses 16 like contempt are organized under an orange colored bar 42b. Positive responses 16 such as surprise, happiness, and joy are organized under a green colored bar 42c.

As shown in FIG. 6b, user 10 engaging with the behavioral tracking program on phone 14, computer, or other electronic device can access and review social media profiles or other media sources that caused a mood decline or negative emotional response. In a profiles caused mood decline report 44, the behavioral tracker program will display pieces of media, like a social media post 46 and its source 36 that caused a negative emotional response from user 10. These pieces of media are collected by the behavioral tracking program through screenshots or pictures that are taken when a negative emotional response from user 10 is detected. The profiles caused mood decline reports are compiled to help the user better understand and identify which types of information, websites, posts 46, or social media platforms 36 cause negative emotional reactions. With this information, user 10 can reduce their frequency of negative emotional reactions by avoiding media platforms 36 that lead to negativity.

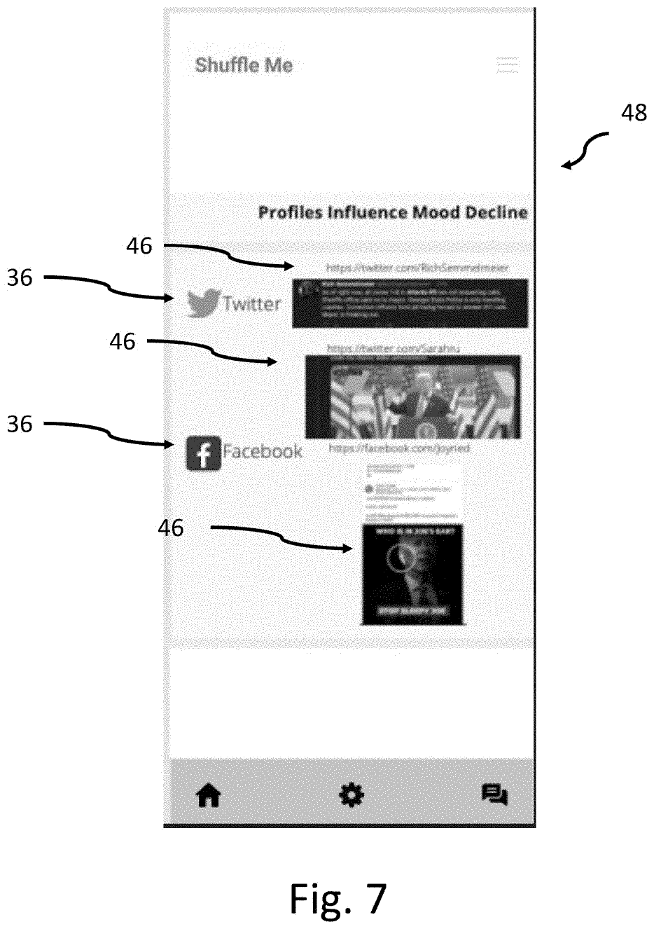

FIG. 7 is an extension of FIG. 6b. As shown in FIG. 7, user 10 engaging with the behavioral tracking program on phone 14, computer, or other electronic device can access and review social media profiles or other media sources that caused a mood decline or negative emotional response. In a profiles influenced mood decline report 48, the behavioral tracking program will display pieces of media, like a social media post 46 and its source 36 that influenced a negative emotional response from user 10. These pieces of media are collected by the behavioral tracking program through screenshots or pictures that are taken when a negative emotional response from user 10 is detected. The profiles influenced mood decline reports are compiled to help the user better understand and identify which types of information, websites, posts 46, or social media platforms 36 influence negative emotional reactions. With this information, user 10 can reduce their frequency of negative emotional reactions by avoiding media sources that lead to negativity.

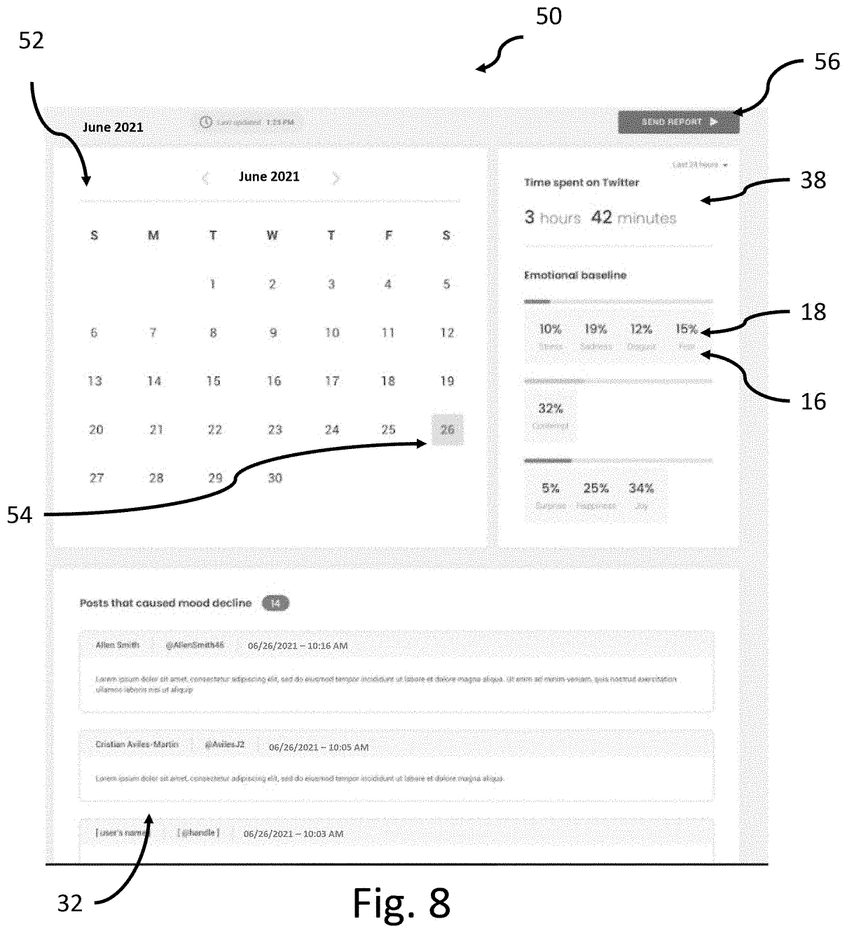

As shown in FIG. 8, an interactive dashboard displays a monthly report 50 with a monthly calendar 52. It shows on a given date 54, the user's emotional baseline 16, emotional percentage 18, time 38 spent within a giving social media platform 36, and social media posts 46 that caused the user's mood to decline. For example, as shown in FIG. 8, user 10, on the example date spent 3 hours and 42 minutes on Twitter. An emotional baseline 16 indicates that the emotions are grouped by their positive, negative, and neutral divided by the percentages of how much the particular platform gives the users positive, negative, and neutral feelings. Under social media posts 46 that caused the user's mood to decline, it show the number of posts that caused the decline, the username, and the timestamp of the mood decline. With the information, user 10 can choose to send the report with to a number of individuals by pressing button 56 and entering the individual's contact information, unfollow specific accounts, or take any additional actions.

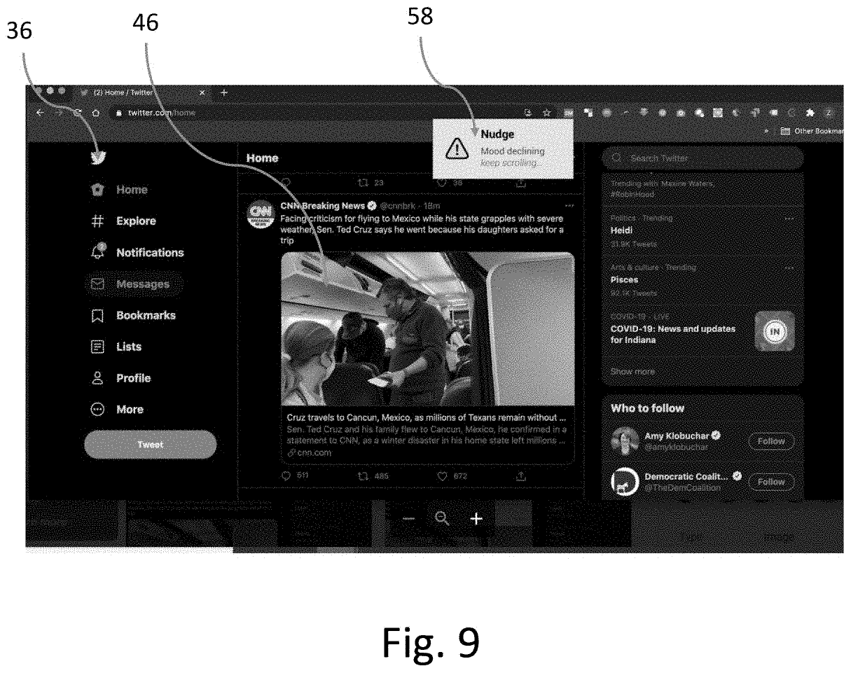

As shown in FIG. 9, a warning notification 58 is sent to user 10 while using social media platform 36 or any other media platform such as online newspapers, chatrooms, etc. by the behavior tracker to alert the user to avoid a social media post 46 because it produced negative feedback scores to the user. For example, as shown in FIG. 9, the user received warning 58 while using a social media platform 36 that the post caused the user's mood to decline. With the information, the user can decide whether continue reading or looking at the content or avoiding it.

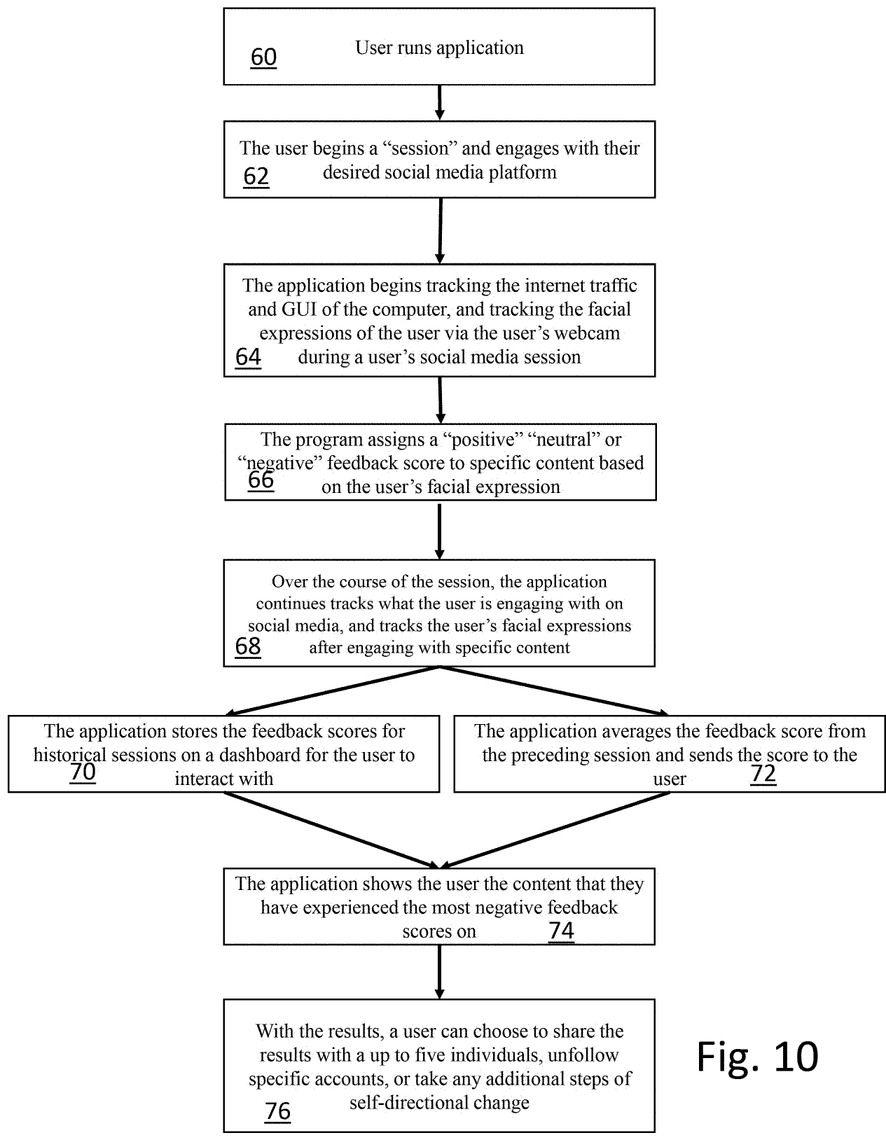

As shown in FIG. 10, user 10 runs the behavioral tracker in start-up step 60. In engagement step 62, as user 10 engages with the session and their desired media and the tracker will detect changes in user's 10 expressions. In initial tracking step 64, the behavior tracker application begins tracking the interact traffic on the electronic device, and tracks the facial expressions of user 10 via user's 10 webcam during a media session. In an emotion assignment step 66, the tracker assigns a positive, neutral, or negative feedback score to specific content 46 based on user's 10 facial expressions and then continues tracking and assigning in monitoring step 68. The behavior tracker application stores the data. In averaging, storing, and displaying steps 70, 72, 74 the behavior tracker application averages the feedback scores and compares user's 10 current session with preceding sessions and sends the scores to user 10 as discussed above. The tracker creates a report that user 10 can review and send in sharing step 76.

Those having ordinary skill in the art will recognize that numerous modifications can be made to the specific implementations described above. The implementations should not be limited to the particular limitations described. Other implementations may be possible.

1. A method for determining emotional states when interacting with media including the steps of
tracking facial expressions of a user of a media,
assigning an emotional score to the tracked tracked facial expressions, and
indicating negative reactions with the media based on the emotional score.
